(19) United States
(12) Patent Application Publication
(10) Pub. No.: US 2023/0313263 A1
(43) Pub. Date: Oct. 5, 2023

(54) ENZYME ACTIVITY ASSAY SYSTEMS AND METHODS

(71) Applicant: GLYCOSPOT APS, Søborg (DK)

(72) Inventors: Julia SCHÜCKEL, Dresden (DE); Robert BAYER, Søborg (DK); Raimon PARÉS VIADER, Søborg (DK); Lennart HANSEN, Søborg (DK); Stjepan Kresimir KRACUN, Søborg (DK); Roberto Thomas LACENTRA, Søborg (DK); Troels Balmer CHRISTENSEN, Søborg (DK)

(21) Appl. No.: 18/004,114

(22) PCT Filed: Jun. 25, 2021
(86) PCT No.: PCT/DK2021/050210

§ 371 Date: Jan. 3, 2023

(30) Foreign Application Priority Data
Jul. 3, 2020 (DK) PA 2020 00798

(51) Int. Cl. G01N 21/78; C12Q 1/40; G16C 99/00

(52) U.S. Cl. CPC C12Q 1/40; G01N 21/78; G16C 99/00

(57) ABSTRACT

A method and a system of determining activity of a target enzyme of a sample is described. The method comprises bringing the sample in physical contact with a substrate for the target enzyme, incubating the sample with the substrate for an actual incubating time, determining a parameter value associated to enzymatic actions of the target enzyme involving the substrate, correlating the actual incubation time for the sample to at least two sets of reference data, determining a best fit standard curve correlated to the actual incubation time for the sample, and correlating the determined parameter value to the best fit standard curve and determine the target enzyme activity. Each of the at least two sets of reference data comprises data representing a standard curve for the parameter associated to the enzymatic actions of the target enzyme involving the substrate as a function of enzyme activity and correlated to an incubation time.

### TECHNICAL FIELD

The invention relates to enzyme activity assay systems and methods of performing an enzyme activity assay for determining enzyme activity in a sample.

### BACKGROUND ART

Enzymes constitute a family of proteins involved in catalyzing chemical reactions within living organisms. As a result of their importance, there are numerous of situations in which it is necessary and/or beneficial to measure enzyme levels, and importantly, enzyme activity.

Within the enzyme family, there are many classes of enzymes that act by facilitating substrate cleavage, for example through hydrolysis or elimination. Such substrate cleavage is usually referred to as substrate degradation and/or digestion.

WO 2016/185533 discloses a method of optical determination of enzyme activity using a biopolymer substrate comprising chemically bonded or entrapped dye. The method may be carried out without allowing a full degradation of the substrate, however this requires that the incubation time used when performing the method should be very accurate according to a preselected incubation time.

Enzymes that degrade or modify polysaccharides are widespread in pro- and eukaryotes and have multiple biological roles and biotechnological applications. Recent advances in genome and secretome sequencing, together with associated bioinformatic tools have enabled large numbers of carbohydrate acting enzymes to be putatively identified. However, there is a paucity of methods for rapidly determination of the biochemical activities of these enzymes.

For many processes, it is desired to know the enzyme activity in seeds and grains, such as cereals, nuts, legumes and spices. Determination of enzyme activity in solid biologic substances is usually performed by extracting the enzyme from the solid biologic substances and determining the enzyme activity in the liquid extract.

The enzyme activity of an enzyme is not directly proportional to the content of the enzyme. Many factors influence the activity of an enzyme. Many of these are known and include for example pH value, temperature, inhibitors and other environmental factors. However, small chemical variations of the enzymes also have large effect and therefore it is necessary to determine the activity of the enzyme and not only the concentration.

Photometric enzyme activity assay, generally requires that a calibration curve generated at a specific reaction time (incubation time) is provided and that the assay is performed with the exact incubation time as the specific reaction time.

In US 2015/0046114 an optimized photometric method is described where there are generated a first calibration curve at a first wavelength and at a first reaction time (suitable for use where target enzyme concentration is low) and a second calibration curve at a second wavelength and at a second reaction time longer than the first reaction time (suitable for use where the concentration of target enzyme is high). In use the enzyme activity assay is carried out to both the first reaction time and the second reaction time and thereafter either the first or the second calibration curve is selected for quantification of the specific analyte based on one or more threshold values.

US5047351 describes a photometric assay where a plurality of calibration curves at different selected reaction times are generated based on reflection optical density. Thereafter in use the assay is performed by allowing the reaction to a first reaction time, which is one of the selected reaction times and measuring the reflection optical density and if it is lower than a predetermined critical value, selecting the calibration curve with the corresponding selected reaction time. If the measured reflection optical density exceeds the predetermined critical value, continuing the reaction further to another of the selected reaction times and repeating until the predetermined critical value is lower than a predetermined critical value and applying the calibration curve with the corresponding reaction time.

### DISCLOSURE OF INVENTION

An object of the present invention is to provide an improved method of a system for determining activity of a target enzyme with a desired high accuracy.

In an embodiment, it is an objective of the invention to provide a method and a system for determining activity of a target enzyme which method and system provides a very fast and reliable determination, which in addition advantageously may be applied with a high flexibility by a user.

In an embodiment, it is an objective of the invention to provide a method and a system for determining activity of a target enzyme, which method and system may be applied with a high flexibility by a user and wherein the target enzyme has a degradative enzyme activity raised for at least one biopolymer.

These and other objects have been solved by the inventions or embodiments thereof as defined in the claims and as described herein below.

It has been found that the inventions or embodiments thereof have a number of additional advantages, which will be clear to the skilled person from the following description.

In another aspect, the invention comprises a system for determining activity of a target enzyme, wherein the target enzyme in principle may be any target enzyme capable of being contained in a sample for being subjected to a substrate for the target enzyme.

The system for determining activity of a target enzyme in a sample, the system comprises
· a substrate for the target enzyme,
· an incubator for incubating the sample with the substrate,
· a reader for determining a parameter associated to enzymatic actions of the target enzyme involving the substrate, and
· a computer system

The computer system is storing at least two sets of reference data. Each set of reference data comprises data representing a standard curve for the parameter associated to the enzymatic actions of the target enzyme involving the substrate as a function of enzyme activity and correlated to an incubation time and an incubation time attribute representing the incubation time, wherein the at least two sets of reference data having different incubation time attribute representing different incubation time.

The computer is advantageously configured for generating a best fit standard curve for any incubating times within an operating range comprising the different incubating times of the reference curves.

It has been found that the system for determining activity of a target enzyme of the invention is very effective, provides surprisingly accurate determinations and allows a highly increased flexibility for the user, since the user does not need to apply an accurate incubation time. For a specific assay the incubation may for example be set to be at least a preselected number of minutes or within a range of minutes and the user may apply any actual incubation time fulfilling this requirement. This makes the system very flexible and ensures the user (e.g. in a laboratory) freedom to perform other tasks during the incubation without risk of errors due to an “inexact” incubation time.

The parameter associated to the enzymatic actions of the target enzyme involving the substrate is in the following also called “the signal parameter” and a specific parameter has a parameter value.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other stated features.

Reference made to “some embodiments” or “an embodiment” means that a particular feature, structure, or characteristic described in connection with such embodiment(s) is included in at least one embodiment of the subject matter disclosed. Thus, the appearance of the phrases “in some embodiments” or “in an embodiment” in various places throughout the specification is not necessarily referring to the same embodiment(s). Further, the skilled person will understand that particular features, structures, or characteristics may be combined in any suitable manner within the scope of the invention as defined by the claims.

The term “substantially” should herein be taken to mean that ordinary product variances and tolerances are comprised.

Throughout the description or claims, the singular encompasses the plural unless otherwise specified or required by the context.

All features of the invention and embodiments of the invention as described herein, including ranges and preferred ranges, may be combined in various ways within the scope of the invention, unless there are specific reasons not to combine such features.

The term “incubation” means to provide an enzymes sample in contact with a substrate to allow enzyme activity, such a degradation/digestion of optional enzyme of the sample.

The term “incubation time” means the time of incubation. I.e. the time of allowing the sample in contact with the substrate until the sample is taken away from the substrate sample or the enzyme activity is stopped by other means e.g. by adding a stop agent, such as sodium carbonate solution. The terms “reaction time” and “incubation time are used interchangeably”.

A stop agent is an agent that is terminating the enzyme activity of the enzyme e.g. by changing pH level.

Each data set of reference data may for example comprise a number of data-pairs of known target enzyme activity and signal parameter and each data set may comprise at least two, preferably three or more data-pairs with different known target enzyme activity.

The reference data sets may be provided by testing a number of samples with different known target enzyme activity, to provide the signal parameter for a number of selected incubation times for each sample with different known enzyme activity to obtain data points each attributed with a signal parameter (a parameter value), a known target enzyme activity and an incubation time. Thereafter each set of reference data may be assembled e.g. virtually assembled according to their common incubation time.

The enzyme activity of the target enzyme is advantageously determined in enzyme units. Such enzyme units are relative units e.g. defined by the standard curve. The standard curve may e.g. define the enzyme unit as enzyme unit per ml fluid extract sample and/or as enzyme unit per mg sample.

The enzyme units may include standard enzyme assay units.

The enzyme unit may in an embodiment be defined by the user.

The enzyme unit may be an enzyme assay unit specifically linked to a particular enzyme, such as “DU” for α-amylase. In an embodiment the enzyme unit is parts-per million (ppm).

It has been found that the sets of reference data each represent a curve that are substantially and/or practically linear. This makes the required number of standard curves for obtaining a very high accuracy surprisingly low. For some type of assays, two reference curves may be sufficient for obtaining a surprisingly high accuracy.

The incubator may be the container e.g. a vial e.g. a filter vial or a microcentrifuge tube containing the sample and the substrate during the incubation time. The incubator may in addition comprise a shaker configured for shaking the sample container.

The incubation may advantageously be performed at any temperature where the enzyme is active. Advantageously the incubation is performed at a temperature of from about 10° C. to about 50° C., such as from about 20° C. to about 30° C., preferably at room temperature. Advantageously the reference data represents the standard curve for the parameter associated to the enzymatic actions of the target enzyme at the corresponding or same temperature as the incubation temperature of the sample incubation.

In an embodiment, the computer is configured for receiving data representing a determined parameter (i.e. a parameter value) for a sample and receiving data representing an associated incubation time for the sample and wherein the computer is programmed for
· correlating the incubation time for the sample to the at least two sets of reference data and determining data representing a best fit standard curve correlated to the incubation time for the sample,
· correlating the data representing the determined parameter to the data representing the best fit standard curve and determine data representing the target enzyme activity, and
· transmitting the data representing the target enzyme activity to a display.

The best fit standard curve is a calculated best fit standard curve for example obtained by performing a regression, such as a linear regression between two or more of the sets of reference data stored on the memory.

Since the reference standard curve most often are practically linear the best fit standard curve may be determined with very high accuracy.

The invention has the benefit that the user need not use a preselected incubation time, but may simply determine which incubation time has been used and feed this used associated incubation time to the computer system. The used associated incubation time may for example be fed to the computer system as the final associated incubation time or it may be fed to the computer system by feeding a start time of the incubation and a stop time of the incubation time. The stop time of incubation may e.g. be fed to the computer system by inserting a container with the incubated sample into the reader.

In an embodiment, the computer system comprises processor unit(s) integrated in the reader and a tablet, wherein the user is entering the start of incubation time and stop of incubating time into the tablet.

The target enzyme may as mentioned be practically any target enzyme capable of interacting with a substrate to provide a readable parameter associated to the enzymatic actions of the target enzyme involving the substrate.

Examples of the target enzymes includes a Hydrolase, a Lyase, a Ligase, a Lipase, a protease, a cellulose and/or an amylase.

In an embodiment, the target enzyme is a target enzyme of a food, such of a vegetable or an animal food. In an embodiment, the target enzyme is an enzyme of grain. The target enzyme may e.g. be extracted as described in copending PCT application requesting priority from same patent application as the present application.

Where the target enzyme is from a solid material, it is desired that the target enzyme is extracted to provide the sample as a liquid sample.

In an embodiment, the target enzyme is a degradative enzyme capable of degrading and/or digestion the substrate, such as glycoside hydrolase (e.g. amylase), protease, lipases and/or cellulase, amylase

The substrate may in principle be any substrate suitable for the selected target enzyme. In an embodiment, the substrate comprises a biopolymer such as a polymer comprising biomolecules selected from polynucleotides, polypeptides, polysaccharides or any combinations thereof, wherein the biopolymer optionally is crosslinked.

In an embodiment, the substrate is a dyed substrate, a chromogenic substrate and/or a chromophore labelled substrate.

The substrate is advantageously a dyed and/or chromogenic substrate.

The dyed and/or chromogenic substrate is advantageously specific for the target enzyme. A specific substrate should in this context be interpreted to be a substrate that is exclusively digested by the target enzyme and not digestible by other enzymes present in the fluid extract sample.

The selected dyed and/or chromogenic substrate may be a gelled biopolymer substrate comprising cross-linked polymeric biomolecules selected from polynucleotides, polypeptides, polysaccharides or any combinations thereof, preferably the dyed and/or chromogenic substrate is an aerogel or a xerogel.

In an embodiment, the substrate is a substrate as described in WO2016/188533.

Where a dyed substrate is used, it is desired that the method comprises filtering of solid parts of the substrate after digestion and prior to determining the absorbance parameter of dye or dye bound to small degraded substrate fragments released from the substrate into the fluid extract sample.

The dye released to the fluid extract sample may be in the form of non-bonded dye incased in a crosslinked polymer or preferably in the form of dye bound to short fragments of the substrate, which have been digested by the target enzyme.

It has been found to be very effective to terminate the incubation time by filtering of the solid, non-dissolved parts of the substrate, to thereby ensure that no further digestion takes place such that no further release of dye occur. The digestion is terminated immediately and thereby the incubation time may be effectively controlled.

Where a chromogenic substrate is used the target enzyme provides that a colored reaction product is formed from the chromogenic substrate.

The chromogenic substrate may be a dissolvable chromogenic substrate, such as a colorless chromogenic substrate. Where a chromogenic substrate is used, it is generally desired to omit filtering, since this otherwise may be a superfluous step.

In an embodiment, the incubation time may be terminated by adding a stop agent.

The parameter may for example be an electrically readable parameter or an optically readable parameter.

A determined parameter is also referred to as a determined parameter value. The parameter may advantageously be an absorbance parameter.

The determining of the target enzyme activity may advantageously comprises measuring the absorbance parameter of the reference sample or sample with released dye or formed colored reaction product provided during the incubation, wherein the absorbance parameter comprises at least one wavelength absorbable by the dye.

In an embodiment, the parameter is an optical parameter and the reader is an optical reader, such as a spectroscope.

The absorbance parameter may be any kind of absorbance parameter, such as, a measure of reflected (not-absorbed) light, a measure of transmitted (not-absorbed) light, a measure of total intensity, a measure of intensity of one or more wavelengths, a measure of intensity profile (intensity as a function of wavelengths) and/or a combination comprising at least one of these.

The absorbance parameter may be determined by a spectroscope comprising a light source and an optical reader.

The light source used for performing the determination of the absorbance parameter, may be any light source comprising at least one wavelength absorbable by the dye. Advantageously the light source comprised a xenon light source, a xenon-mercury light source and/or a diode. A xenon light source or a xenon-mercury light source may be especially preferred where a broad wavelength range is desired e.g. covering UV to IR, such as in the range of 185-2000 nm.

The reader for reading the absorbance parameter may be any optical reader capable of reading at least a fraction of not absorbed light, preferably comprising at least one wavelength absorbable by the dye.

The enzyme activity of the reference samples is advantageously determined at a selected pH value where the target enzyme is active, preferably in a range of maximal activity.

Advantageously, the computer is configured for receiving at least a portion of the data representing the associated incubating time for the sample from a user via a user interface, such as via screen, keyboards, a mouse or sound (voice).

In an embodiment, the computer system is configured for receiving at least a portion of the data representing the associated incubation time for the sample from the reader. For example the stop time of the incubation time may be the time of providing the incubated sample to the reader.

In an embodiment, the computer system is configured for receiving the data representing the determined parameter value for the sample from a user via a user interface. In an embodiment, the computer system is configured for receiving the data representing the determined parameter value for the sample from the reader. In an embodiment, the reader comprises a computer forming part of the computer system.

Advantageously, the computer system is storing at least three sets of reference data, such as five sets of reference data, each set of reference data comprises data representing a standard curve for the parameter associated to the enzymatic actions of the target enzyme involving the substrate as a function of enzyme activity and correlated to an incubation time and an incubation time attribute representing the incubation time, wherein the respective sets of reference data having different incubation time attribute representing different incubation time.

Advantageously, each set of reference data comprises at least two data-pairs of enzyme activity value versus parameter value, preferably each set of reference data comprises at least three data pairs, such as at least five data pairs, such as at least eight data pairs.

To ensure a large freedom for the user in respect of actual incubation time used and at the same time have a desired high accuracy it is desired that the sets of reference data comprises at least two sets of reference data having different incubation time attribute representing incubation time that differs with at least about 30 seconds, such as at least about 1 minute, such as at least about 5 minutes, such as at least about 10 minutes.

Hence, where the actual used incubation time is within the range of the longest incubation time and shortest incubation time of the reference data, the system of the invention may be extremely accurate. However, it has been found that using an incubation time outside the range of the longest incubation time and shortest incubation time of the reference data, very good enzyme activity determinations may also be provided in a very fast manner.

The invention also comprises a method of determining activity of a target enzyme in a sample, the method comprises
· bringing the sample in physical contact with a substrate for the target enzyme,
· incubating the sample with the substrate for an actual incubating time,
· determining a parameter value associated to enzymatic actions of the target enzyme involving the substrate, and
· correlating the actual incubation time for the sample to at least two sets of reference data,
· determining a best fit standard curve correlated to the actual incubation time for the sample,
· correlating the determined parameter value to the best fit standard curve and determine the target enzyme activity,
wherein each of the at least two sets of reference data comprises data representing a standard curve for the parameter associated to the enzymatic actions of the target enzyme involving the substrate as a function of enzyme activity and correlated to an incubation time and an incubation time attribute.

The actual incubation time may also be referred to as an incubation time associated to the sample in question.

The method may be carried out using the system as described above.

The sample may advantageously be a liquid sample. The substrate may be as described above, the substrate applied in the method may advantageously be identical to the substrate used for generating the reference data sets.

The parameter value may be as described above. The applied parameter is advantageously the same parameter which is applied for the generation of the reference data sets, e.g. absorbance at one or more selected wavelengths.

The enzyme activity of the sample is advantageously determined at a selected pH value where the target enzyme is active, preferably in a range of maximal activity. Preferably same pH value is applied for the activity determination of the sample as applied in reference sample enzyme activity assays.

In an embodiment, the actual incubation time is at least one minute, such as at least 2 minutes, such as 5 to 15 minutes.

In an embodiment, the preselected reference incubation time, for the generation of at least one of the sets of reference curves is from 5 minutes shorter to 5 minutes longer than the actual incubation time.

The Parameter determination for the sample is preferably performed using identical substrate, pH value and parameter as applied for the parameter determination of the reference samples. Advantageously, also the temperature is within a same range of op to 10° C., such as up to 5° C.

The correlation of the actual incubation time for the sample to at least two sets of reference data, may include selecting at least two sets of reference data. The selected sets of data may preferably include a set of reference data correlated to an incubation time which is within 10 minutes from the actual incubation time, such as within 5 minutes from the actual incubation time. In an embodiment, the selected sets of data may preferably include a set of reference data correlated to an incubation time which is larger than the actual incubation time and a set of reference data correlated to an incubation time which is shorter than the actual incubation time.

All features of the inventions including ranges and preferred ranges can be combined in various ways within the scope of the invention, unless there are specific reasons not to combine such features.

### BRIEF DESCRIPTION OF THE EXAMPLES AND DRAWING

The invention is being illustrated further below in connection with a few examples and embodiment and with reference to the drawings in which:

FIG. 1 shows a table of pH optimum for a number of enzymes.

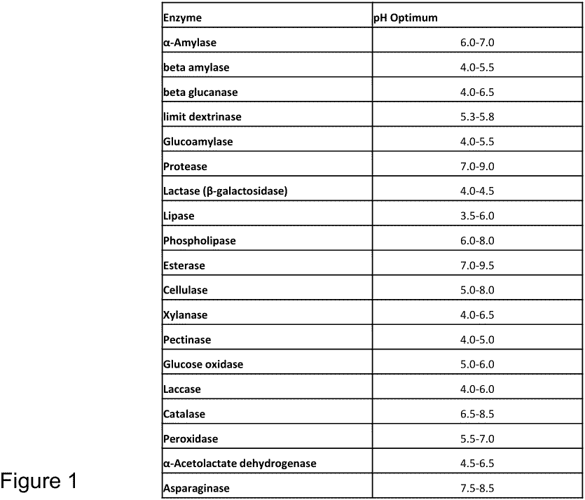

FIG. 2a illustrates a system for determining activity of a target enzyme of a sample.

FIG. 2b illustrates a system for determining activity of a target enzyme of a grain material.

FIG. 3a illustrates a standard curve associated to a preselected extraction and digestion procedure.

FIG. 3b illustrates a further standard curve associated to a preselected extraction and digestion procedure.

FIG. 4a illustrates data points for five standard curves, each associated to respective preselected extraction times for extracting target enzyme from a grain material.

FIG. 4b illustrates five standard curves for five different selected extracting times.

FIG. 5a illustrates data points for six standard curves, each associated to respective preselected reference incubating times for enzymatic actions involving a substrate by respective samples containing known amount of target enzyme.

FIG. 5b show standard curves of concentration as a function of absorbance for a number of incubation times.

FIG. 6 shows determined absorbance parameter values as a function of the Ring trial WK values obtained in example 2 an example 3.

FIG. 7 shows determined absorbance parameter values as a function of the Ring trial WK values obtained in example 4.

FIG. 8 shows determined absorbance parameter values as a function of the Ring trial WK values obtained in example 5.

FIG. 9 shows determined absorbance parameter values as a function of the Ring trial WK values obtained in example 6.

FIG. 10 shows determined absorbance parameter values as a function of the DU values obtained in example 7.

FIGS. 11a and 11b shows determined absorbance parameter values as a function of the BU3/ml and BU3/g values obtained in example 8.

FIGS. 12a and 12b shows determined absorbance parameter values as a function of the BU3/ml and BU3/g values obtained in example 9.

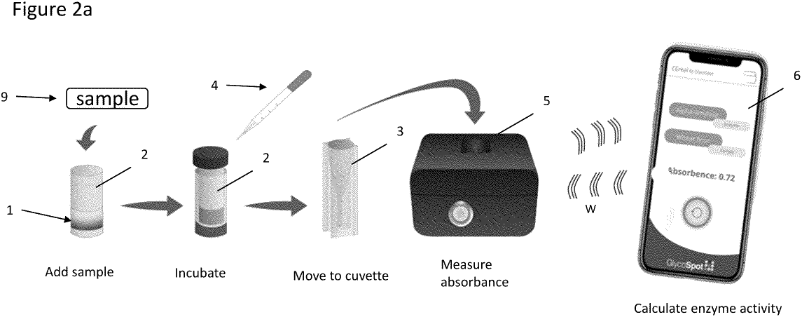

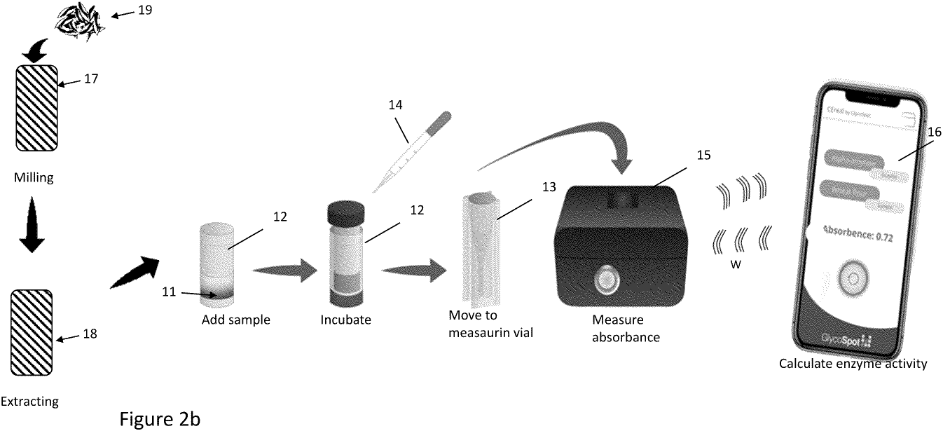

The system illustrated in FIG. 2a for determining activity of a target enzyme of a sample, comprises a container 2 containing a substrate 1, a cuvette 3, a pipette 4, a reader 5 and a tablet 6, such as a smartphone.

In use, a sample 9 comprising a target enzyme is added into the container 2 comprising the substrate for the target enzyme. The sample is advantageously a liquid sample, e.g. obtained from a biological matter, e.g. by extraction, dissolving or diluting or without any pretreatment. The container 2 act as an incubator and the sample is incubated with the substrate for an incubating time, also referred to as an actual incubation time or the incubation time associated to this sample. The incubation time may be terminated by adding a stop agent to the container 2. After incubation, a portion of the sample is taken out from the container 2 using the pipette 4 and added to the cuvette 3. The cuvette 3 is inserted into the reader 5, which in this example is an optical reader configured for reading absorbance. The reader 5 is reading and/or determining an absorbance parameter value associated to enzymatic actions of the target enzyme involving the substrate. The absorbance value is transmitted e.g. wireless as indicated with the waves W to the tablet 6. The tablet may also be configured to transmit data to the reader 5, such as data representing the actual incubation time. The reader 5 may be calibrated e.g. using a blank sample.

A computer of the reader 5 and a computer of the tablet 6 form parts of the computer system and they may be as described above. The computer system comprises a memory storing reference data representing one or more standard curves, such as at least two sets of reference data, each set of reference data comprises data representing a standard curve for said parameter associated to said enzymatic actions of said target enzyme involving said substrate as a function of enzyme activity and correlated to an incubation time and an incubation time attribute representing said incubation time, wherein said at least two sets of reference data having different incubation time attribute representing different incubation time

The system illustrated in FIG. 2b for determining activity of a target enzyme of a grain material, comprises a milling device 17, an extracting device 18, a container 12 containing a substrate1 1, a cuvette 13, a pipette 14, a reader 15 and a tablet 16, such as a smartphone.

In use, a sample of grain material 19 comprising a target enzyme is milled in the milling device 17 e.g. as described above. The milled material or at least a portion of the milled material is moved to the extracting device 18 for extracting target enzyme into an extraction liquid e.g. as described above. At least a portion of the extraction liquid is moved to the container 12 e.g. using a method as described. The container 12 act as an incubator and the extracting liquid with extracted target enzyme is incubated with the substrate for an incubating time, also referred to as an actual incubation time or the incubation time associated to this sample. The incubation time may be terminated by adding a stop agent to the container 12. After incubation, a portion of the liquid is taken out from the container 2 using the pipette 4 and added to the cuvette 13. The cuvette 13 is inserted into the reader 15, which in this example is an optical reader configured for reading absorbance. The reader 15 is reading and/or determining an absorbance parameter value associated to enzymatic digestion of the substrate by the target enzyme. The absorbance value is transmitted e.g. wireless as indicated with the waves W to the tablet 16. The tablet may also be configured to transmit data to the reader 15, such as data representing the actual incubation time. The reader may be calibrated e.g. using a blank sample.

A computer of the reader 15 and a computer of the tablet 16 form parts of the computer system and they may be as described above. The reader 15 and the tablet 16 may be as described for the system of FIG. 2a.

FIG. 2 illustrates a system for determining activity of a target enzyme of a grain material.

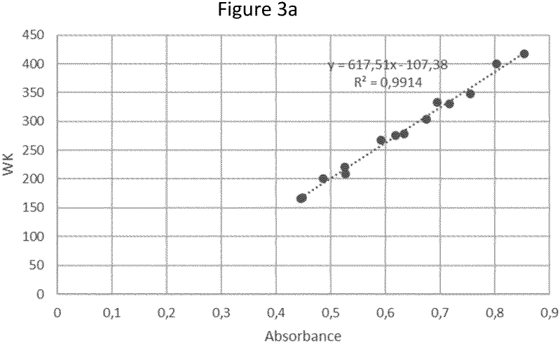

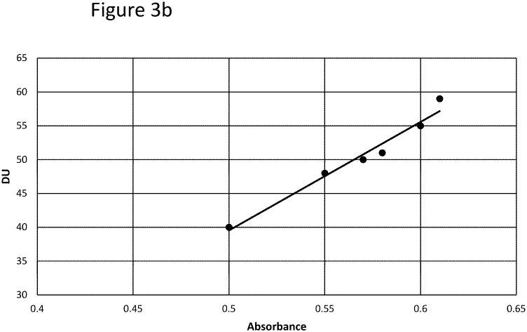

The standard curve associated to a preselected extraction and digestion procedure illustrated in FIG. 3a represents beta-amylase enzyme activity in terms of diastatic power (unit Windisch-Kolbach (WK)) as a function of absorbance parameter at a preselected incubation time. The standard curve may be obtained by determining the absorbance parameter value for a number of samples with known beta-amylase enzyme activity (WK), using the preselected incubation time. Where the beta-amylase enzyme activity is determined in grain, the grain may be extracted as described above using a preselected extraction time.

The standard curve associated to a preselected extraction and digestion procedure illustrated in FIG. 3b and represent alpha amylase enzyme activity in terms of dextrinization units (DU) as a function of absorbance parameter at a preselected incubation time. The standard curve may be obtained by determining the absorbance parameter value for a number of samples with known alpha amylase enzyme activity (DU), using the preselected incubation time. Where the alpha amylase enzyme activity is determined in grain, the grain may be extracted as described above using a preselected extraction time.

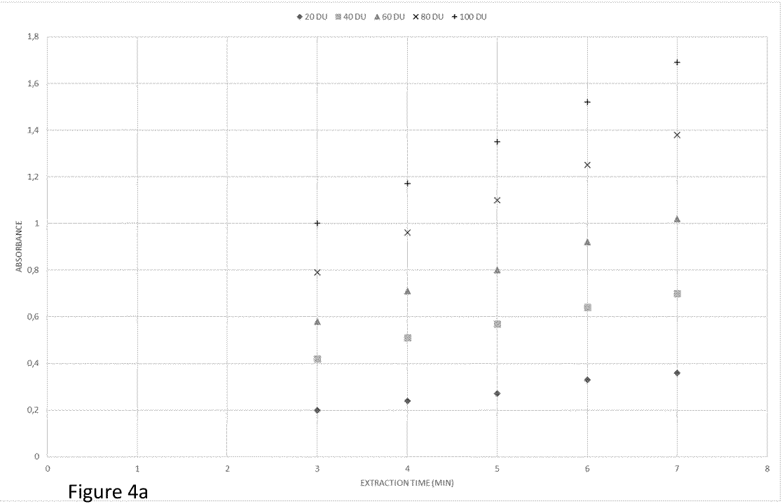

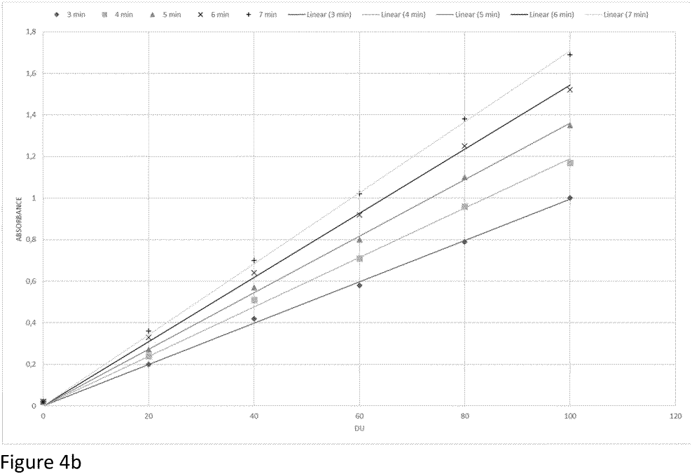

The five standard curves illustrated by the data points in FIG. 4a, show standard curves for alpha amylase enzyme activity in terms of dextrinization units (DU) as a function of absorbance parameter where the standard curves are associated to respective preselected extraction times for extracting target enzyme, here alpha amylase, from a grain material.

The curves may be obtained by determining absorbance parameter values for a number of grain material samples with different and known alpha amylase enzyme activity (DU) and at different extraction times. Preferably, the incubation time used for the determinations are identical. In this example the absorbance parameter values for five grain material samples with different and known alpha amylase enzyme activity (DU) - here 20, 40, 60, 80 and 100 DU respectively were determined at extraction times of 3, 4, 5, 6 and 7 minutes.

It will be observed that the data points for each standard curve are lying on a substantially straight line.

The five standard curves illustrated in FIG. 4b for five different selected extraction times are provided using the data points of FIG. 4a. The five standard curves are each associated to respective preselected reference extracting times for extracting of alpha amylase and show absorbance parameter as a function of extraction time of extracting of alpha amylase from grain.

It can be seen that the standard curves are practically linear, which makes it relatively simple to determining a best fit standard curve for any extraction time, such as an extraction time associated to a sample under examination, where the extraction time differs from the extraction times used in generating the standard curves - e.g. an extraction time between extraction times used in generating the standard curves.

After having determined the best fit standard curve for such sample under examination, the target enzyme activity may be determined from the absorbance parameter value determined for the sample under examination.

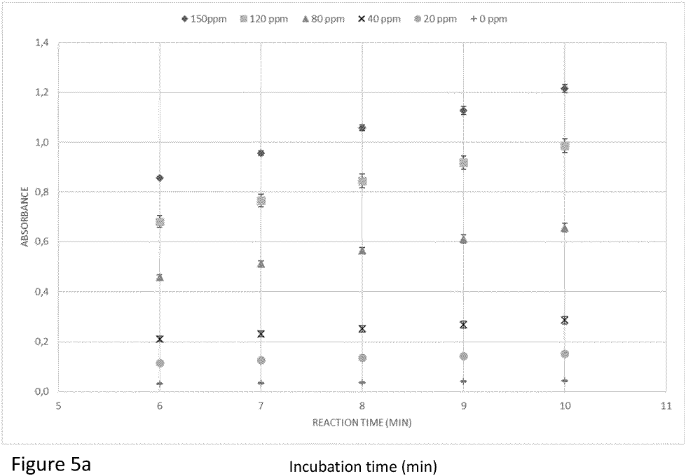

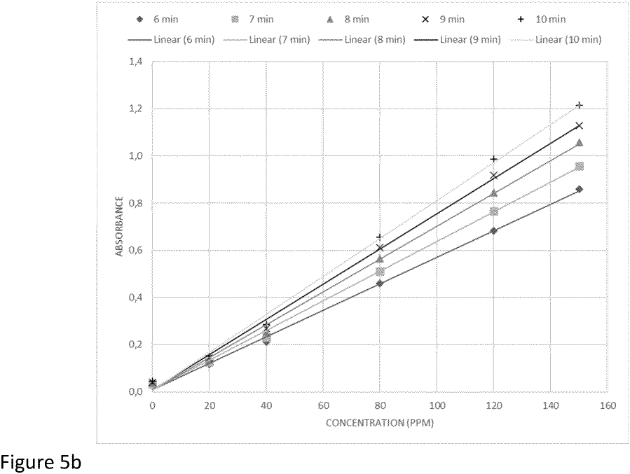

The six standard curves illustrated by data points in FIG. 5a, is each associated to respective preselected reference incubating times for enzymatic actions involving a substrate by respective samples containing known amount in ppm of target enzyme.

The curves may be obtained by determining absorbance parameter values for a number of samples with different and known amounts of target enzyme (ppm) and at incubation times. It will be observed that the data points for each standard curve are lying on a substantially straight line.

The standard curves illustrated in FIG. 5b of concentration as a function of absorbance for a number of incubation times are obtained using the data points of FIG. 5a.

It can be seen that the six standard curves are practically linear, which makes it relatively simple to determining a best fit standard curve for any incubation time, such as an incubation time associated to a sample under examination, where the incubation time differs from the incubation times used in generating the standard curves - e.g. an incubation time between incubation times used in generating the standard curves.

After having determined the best fit standard curve for such sample under examination, the target enzyme activity may be determined from the absorbance parameter value determined for the sample under examination.

FIG. 5a. illustrates six standard curves, each associated to respective preselected reference incubating times for digestion a substrate by a fluid extract sample and representing absorbance parameter as a function of incubation time for samples with known target enzyme activity, wherein the respective preselected incubating times differs from each other with respect to preselected reference incubation time.

FIG. 5b show the concentration as a function of absorbance for a number of incubation times.

In the following examples the terms “Distatic power” and “Ring trial” has the following meaning:

Diastatic power: The malts ability to break down starches into simpler fermentable sugars during the mashing process. It is measured in Windisch-Kolbach (WK), loB or Lintner. By this definition, this is the join action of all amylases, mostly alpha amylase and beta amylase, which are the main sugar producing enzymes during mashing. However, since alpha amylase is always in excess, diastatic power is somehow correlated with beta amylase.

Ring trial: Validation of the methods of one’s lab through the collaboration of typically 10-100 different labs. It includes methods for diastatic power and alpha amylase. The main ring trials in Europe are organized by the Institute Francais de Boissons, de la Brasserie et de la Malterie (IFBM), LGC Standards, known as the Malt analytes scheme (MAPS), and the ones organized by the VLB institute in Berlin.

### EXAMPLE 1

### Constructing a Calibration Curve for Determining Alpha-Amylase Activity in Barley Malt Samples

The construction of calibration curves is based on grain reference samples with known reference values of enzyme activities supplied by recognized certification bodies such as EBC and IFBM.

13 samples barley grain reference samples with known alpha-amylase activity is obtained from the European Brewery Convention and the French Institute of Beverages, Brewing and Malting. The sample is as follows:

| Barley reference sample # | known alpha-amylase activity (DU) | known beta-amylase activity (WK) |
|---|---|---|
| 1 | 59 | 258 |
| 2 | 55 | 595 |
| 3 | 51 | 250 |
| 4 | 48 | 222 |
| 5 | 50 | 390 |
| 6 | 40 | 203 |
| 7 | 55 | 267 |
| 8 | 64 | 271 |
| 9 | 64 | 269 |
| 10 | 66 | 334 |
| 11 | 64 | 331 |
| 12 | 24 | 223 |
| 13 | 49 | 269 |

The dextrinizing units (DU) is a standard unit in the malt industry and specified in EBC Method “4.13 α-AMYLASE CONTENT OF MALT (IM) -2006”. The diastatic power measured in Windisch-Kolbach units is specified in EBC method 4.12.1.DIASTATIC POWER OF MALT BY SPECTROPHOTOMETRY (MANUAL METHOD) - 2018.

A fluid extract reference sample is obtained from each barley malt reference sample using a preselected extraction procedure.

The preselected extraction procedure is as follows:
The samples are milled by use of a lab mill, e.g. Bühler Miag Disc Mill
300 mg of the milled grain material from each barley reference sample is mixed with 50 ml liquid extraction buffer. Each sample of liquid extraction with barley reference sample is arranged in a shaker. Let the enzyme extraction go on for 5 minutes with gentle shaking at 20 rpm using an overhead shaker, for allowing the enzyme extraction for 5 minutes.

The composition of the liquid extraction buffer was a maleic acid based extraction buffer prepared according to the following protocol:

Dissolve 134.1 g of malic acid, 70 g of NaOH, and 58.4 g of NaCl in 900 mL of water (deionized or ultra-pure). Next, add 6.0 g CaCl₂*H₂O until complete dissolution. The pH should be adjusted to pH 5.4 by dropwise addition of concentrated (4 mol L⁻¹) NaOH or HCl. Sodium azide (1.0 g) can be added as a preservative (antimicrobial agent). If sodium azide is added, the reagent will be stable for more than one year. If not, the shelf-life of the reagent will be 2 weeks if preserved at 4° C. This concentrated buffer should be diluted 200 times (e.g. 50 mL of concentrated solution for a final volume of 1000 mL) in order to be used in the extraction protocol.

The extraction was performed at room temperature (21° C.).

Thereafter a fluid extract reference sample of 250 microliter is taken of each liquid extraction buffer with extracted enzyme using a pipette suitable for the purpose. Each sample was carefully aspirated from the liquid phase and thereby minimizing the risk of introducing solid matter in the pipette tip. After proper aspiration, the sample was dispensed in the provided filter vial containing the substrate and further diluted by adding 250 microliter of the above mentioned buffer.

The substrates were produced by first dyeing polysaccharides with one of the four chlorotriazine dyes (red, blue, green or yellow) via nucleophilic aromatic substitution. The polysaccharides were then cross-linked with 1,4-butanediol diglycidyl ether via base-catalysed epoxide opening. The resulting materials are hydrogels, which can be easily dispensed using syringes into 96-well filter plates or vials. Additional info about production of such substrate may be found in Kračun, S.K., et al., A new generation of versatile chromogenic substrates for high-throughput analysis of biomass-degrading enzymes. Biotechnology for Biofuels, 2015. 8(1): p. 70.

The alpha-amylase in the sample will digest the substrate and develop a blue colored solution with an intensity related to the enzymatic activity as a function of incubation time.

The incubation was performed at room temperature applying a reference incubating time of 5 minutes with gentle shaking at 20 rpm using an overhead shaker.

For each sample, the reference incubation time is terminated by suction of the fluid extract reference sample with released substrate fragments and dye over the filter in each container and collecting the fluid extract reference sample in a cuvette.

A further cuvette is supplied by a “blind” sample of the liquid extraction buffer without any extracted enzyme. Instead of a blind sample of liquid extraction buffer, a blind sample of pure water could have been used.

### Measurement

Each reference sample including the blind is subjected to a spectrometer to determine the intensity of transmitted light of a light source comprising a wavelength absorbable by the dye.

An absorbance parameter is determined for each reference samples by withdrawing the light intensity transmitted through the blind from the light intensity transmitted through the respective samples.

Thereafter the respective absorbance parameter is plotted as a function of alpha amylase activity (DU).

| Barley reference sample # | known alpha-amylase activity (DU) | absorbance (# transmitted light intensity - blind transmitted light intensity) |
|---|---|---|
| 1 | 59 | 1.22 |
| 2 | 55 | 1.2 |
| 3 | 51 | 1.16 |
| 4 | 48 | 1.1 |
| 5 | 50 | 1.14 |
| 6 | 40 | 1.0 |

The absorbance is determined according to the formula:

$$A = {log}_{10} \frac{{\text{Φ}}_{\text{e}}^{\text{i}}}{{\text{Φ}}_{\text{e}}^{\text{t}}} = - {log}_{10} T ,$$

where

$${\text{Φ}}_{\text{e}}^{\text{t}}$$

is the radiant flux transmitted by that material,

$${\text{Φ}}_{\text{e}}^{\text{i}}$$

is the radiant flux received by that material,

$$T = {\text{Φ}}_{\text{e}}^{\text{t}} / {\text{Φ}}_{\text{e}}^{\text{i}}$$

is the transmittance of that material.

### Example 2

### Ring Trial Validation - Extraction Time

Ring trial validations using five different milled malt samples having different WK values were performed.

The five samples of milled barley malt with different and known WK value were as follows:

| Sample | Sample 1 | Sample 2 | Sample 3 | Sample 4 | Sample 5 |
|---|---|---|---|---|---|
| WK | 203 | 267 | 334 | 379 | 60 |

The samples were tested according to the following protocol:
Weigh 200 mg of milled malt.
Extraction with extraction buffer for 10 minutes at 35° C.
Syringe filtration with 0.45 µm filter. Collect the filtrate.
Incubation of 250 µL filtrate with substrate for an incubation time at 35° C.
Stop the enzymatic cleaving by adding a stopping reagent
Read the resulting liquid at 410 nm in a spectrophotometer.

The extraction buffer used was the maleic acid based extraction buffer described in example 1. For each sample, 25 mL of extraction buffer was used together with 35 mg Dithiothreitol.

The substrate used was a glucose-based oligosaccharide conjugated with a chromophore, able to absorb visible light at 410 nm when the pH is higher than 9, mixed with a glucosidase. By the action of the enzyme of interest, in this case beta amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™ .

The absorbance parameter for each sample was determined by withdrawing the light intensity transmitted through a blind from the light intensity transmitted through the respective samples.

Each test was performed in triplicate and the average absorbance parameter values were determined.

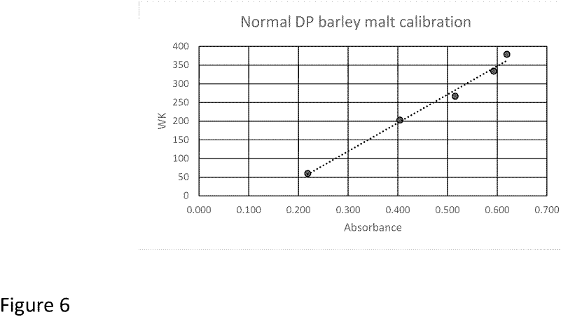

Thereafter the respective absorbance parameter values were plotted as a function of the Ring trial WK values. The plot is shown in FIG. 6.

The result indicates a good correlation with the ring trial samples even with a relatively low extraction time, here 10 minutes. It is assessed that an even lower extraction time could have been applied and still maintaining a good correlation with the ring trial.

### Example 3

### Reference Curve for Barley Malt - Diastatic Power Range 30-500 WK

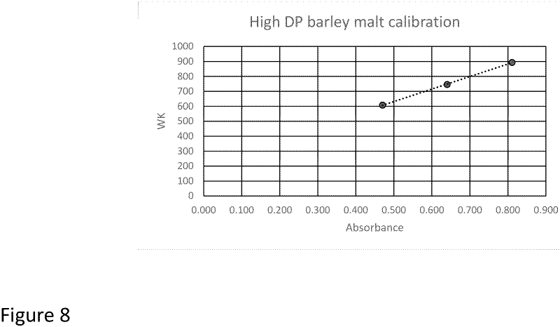

The test results obtained in example 2 and shown in FIG. 8 were applied as reference curve for barley malt.

The tests are repeated using different incubation times including the incubation times 3 minutes, 10 minutes and 20 minutes to thereby prepare additional sets of reference data correlated to respective incubation times as described above.

### Example 4

### Ring Trial Validation - Incubation Time

Ring trial validations using three different milled malt samples having different WK values were performed.

The samples were tested according to the protocol given in example 2, where the buffer, the substrate, the stopping agent and the reading out were as in example 2.

Each sample was tested with three different incubation times, namely 3 minutes, 5 minutes and 7 minutes respectively.

Each test was performed in triplicate and the average absorbance parameter values were determined.

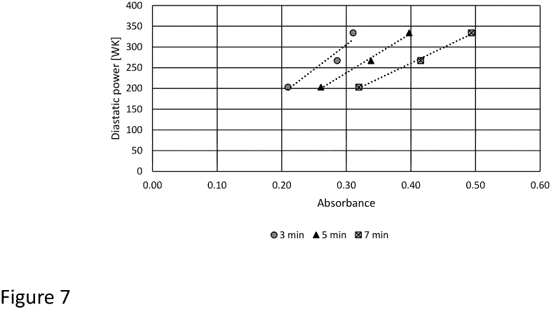

Thereafter the respective absorbance parameter values were plotted as a function of the Ring trial WK values. The plot is shown in FIG. 7.

The results show a very good correlation with the ring trial samples for each of the three incubation times

Thus, by applying data pairs of the WK-values/ determined absorbance parameter values as reference data, the computer system may be programmed to determine the WK value for an unknown sample tested according to the protocol and with an incubation time which may be any time within a range 3-7 minutes and at least some minutes beyond the 3-7 minutes incubation time.

### Example 5

### Preparing Reference Curve for Barley Malt - Diastatic Power Range 500-100 WK

Three samples of milled barley malt with different and known WK value were tested:

| Sample | Sample 1 | Sample 2 | Sample 3 |
|---|---|---|---|
| WK | 893* | 608 | 746 |

The samples were tested according to the protocol given in example 2, where the buffer, the substrate, the incubation time, the stopping agent and the reading out were as in example 2.

*The actual WK value of the sample 1 material was 595, however, for test of this sample 50% more barley malt (i.e. 300 mg) was used to represent the 895 WK value.

Each test was performed in triplicate and the average absorbance parameter values were determined.

Thereafter the respective absorbance parameter values were plotted as a function of the Ring trial WK values. The plot is shown in FIG. 8.

The tests may be repeated using different incubation times. For providing two or more sets of reference data correlated to respective incubation times as described above.

### Example 6

### Preparing Reference Curve for Wheat Malt - Diastatic Power Range 200-500 WK

Four samples of milled wheat malt with different and known WK value were tested:

| Sample | Sample 1 | Sample 2 | Sample 3 | Sample 4 |
|---|---|---|---|---|
| WK | 223 | 269 | 390 | 445 |

The samples were tested according to the protocol given in example 2, where the substrate, the incubation time, the stopping agent and the reading out were as in example 2.

The extraction buffer was as in example 2 but the amount applied was 50 mL together with 70 mg Dithiothreitol/sample.

Each test was performed in triplicate and the average absorbance parameter values were determined.

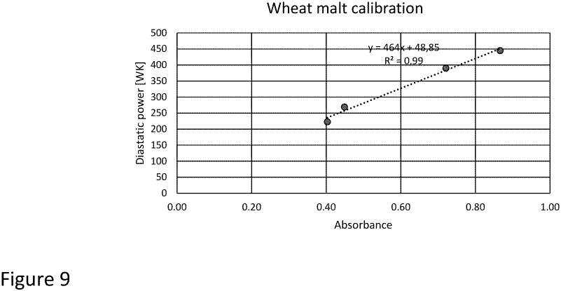

Thereafter the respective absorbance parameter values were plotted as a function of the Ring trial WK values. The plot is shown in FIG. 9.

The tests may be repeated using different incubation times. For providing two or more sets of reference data correlated to respective incubation times as described above

### Example 7

### Preparing Reference Curve for Alpha Amylase Activity for Barley Malt (10-100 DU)

Three different samples of barley malt with known DU values were subjected to an extraction according to the following extraction protocol:
Weigh 200 mg of milled malt.
Extraction with extraction buffer for 10 minutes at 35° C.
Syringe filtration with 0.45 µm filter. Collect the filtrate.

The three samples were subjected to a dilution series to provide in total 9 diluted samples, three from each of the samples A, B and C as listed below:

| Sample | mL buffer 3 —dilution step | Dilution ratio (always pipette 250 µL of the extraction) | Known and calculated-DU |
|---|---|---|---|
| Sample A 3 - 51 DU | 45 | 181 | 10,4 |
| | 9 | 37 | 51 |
| | 4,5 | 19 | 99,3 |
| Sample B 6-40 DU | 36 | 145 | 10,2 |
| | 12 | 49 | 30,2 |
| | 9 | 37 | 40,0 |
| Sample C 10 - 65,6 DU | 27 | 109 | 22,3 |
| | 9 | 37 | 65,6 |
| | 7 | 29 | 83,7 |

The samples were tested according to the following protocol:
Incubation of 250 µL filtrate with substrate for an incubation time at 35° C.
Stop the enzymatic cleaving by adding a stopping reagent
Read the resulting liquid at 410 nm in a spectrophotometer.

The buffer used for the dilution series was the maleic acid based extraction buffer described in example 1.

The substrate used was a glucose-based oligosaccharide conjugated with a chromophore, able to absorb visible light at 410 nm when the pH is higher than 9, mixed with a glucosidase. By the action of the enzyme of interest, in this case alpha amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™ .

The absorbance parameter for each sample was determined by withdrawing the light intensity transmitted through a blind from the light intensity transmitted through the respective samples.

Each test was performed in triplicate and the average absorbance parameter values were determined.

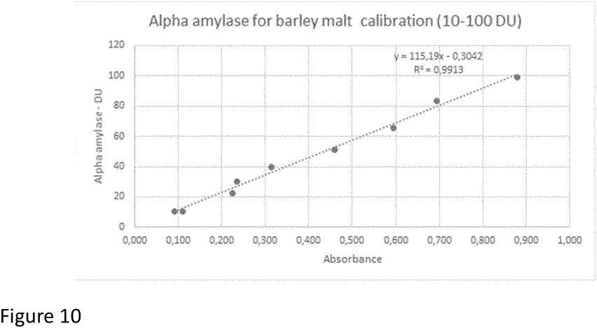

Thereafter the respective absorbance parameter values were plotted as a function of the DU values. The plot is shown in FIG. 10.

The tests may be repeated using different incubation times. For providing two or more sets of reference data correlated to respective incubation times as described above.

### Example 8

### Preparing Reference Curve for Beta Amylase Activity in Malt in Betamyl-3 Units BU3

Betamyl-3 unit is the typical unit for beta amylase activity.

A single sample of barley malt with known BU3 value was subjected to an extraction according to the following extraction protocol:
Weigh 200 mg of milled barley malt.
Extraction with extraction buffer for 10 minutes at 35° C.
Syringe filtration with 0.45 µm filter. Collect the filtrate.

The sample were subjected to a dilution series to provide in total 6 diluted samples.

Each of the 6 dilution samples was tested according to the following protocol:
Incubation of 250 µL filtrate with substrate for an incubation time at 35° C.
Stop the enzymatic cleaving by adding a stopping reagent
Read the resulting liquid at 410 nm in a spectrophotometer.

The buffer used for the dilution series was the maleic acid based extraction buffer described in example 1.

The substrate used was a glucose-based oligosaccharide conjugated with a chromophore, able to absorb visible light at 410 nm when the pH is higher than 9, mixed with a glucosidase. By the action of the enzyme of interest, in this case beta amylase, the oligosaccharide is then accessible to the glucosidase, which then makes the chromophore free of glucose molecules. Such chromophore would then change color by the addition of the stopping reagent, and its intensity will depend directly on how much the enzyme of interest cleaves the oligosaccharide.

The incubation time used was 7 minutes.

The stopping agent used was an alkaline sodium carbonate solution.

The spectrophotometer used was the spectrometer sold by Glycospot under the trade name SIRIUS™ .

The absorbance parameter for each sample was determined by withdrawing the light intensity transmitted through a blind from the light intensity transmitted through the respective samples.

Each test was performed in triplicate and the average absorbance parameter values were determined.

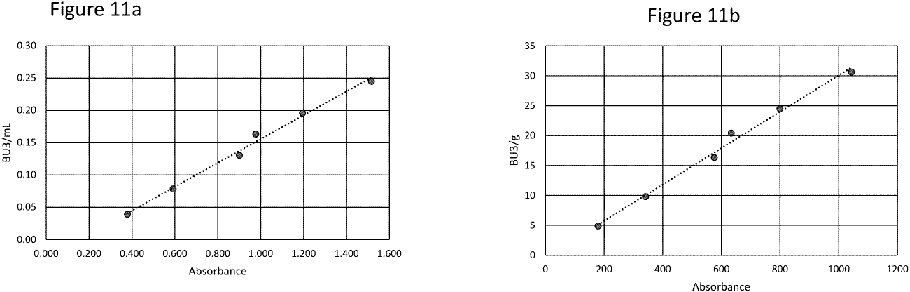

Thereafter the respective absorbance parameter values were plotted as a function of the BU3 values. The plot is shown in FIGS. 11a and 11b. FIG. 11a shows the respective absorbance parameter values versus the BU3/mL value and FIG. 11b shows the respective absorbance parameter values versus the BU3/g value.

The tests may be repeated using different incubation times. For providing two or more sets of reference data correlated to respective incubation times as described above.

### Example 9

### Preparing Reference Curve for Beta Amylase Activity in Malt Extract

The test of example 8 was repeated but with the difference that on 1 g of milled barley malt was extracted using 25 mL extraction buffer and wherein the dilution series was providing 4 diluted samples.

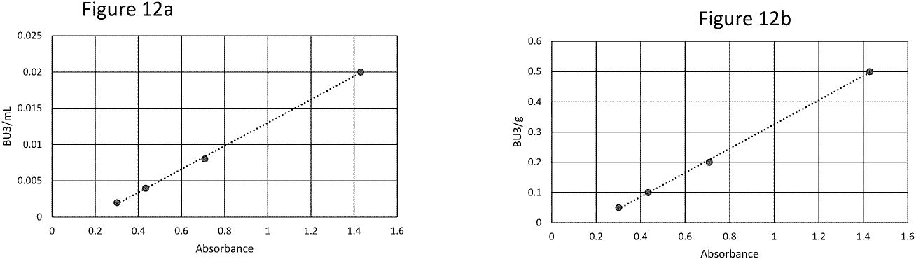

The respective absorbance parameter values were plotted as a function of the BU3 values. The plot is shown in FIGS. 12a and 12b. FIG. 12a shows the respective absorbance parameter values versus the BU3/mL value and FIG. 12b shows the respective absorbance parameter values versus the BU3/g value.

The tests may be repeated using different incubation times. For providing two or more sets of reference data correlated to respective incubation times as described above.

1. A system for determining activity of a target enzyme in a sample, the system comprises
a substrate for the target enzyme,
an incubator for incubating said sample with said substrate,
a reader for determining a parameter associated to enzymatic actions of said target enzyme involving said substrate, and
a computer system
wherein the computer system is storing at least two sets of reference data, each set of reference data comprises data representing a standard curve for said parameter associated to said enzymatic actions of said target enzyme involving said substrate as a function of enzyme activity and correlated to an incubation time and an incubation time attribute representing said incubation time, wherein said at least two sets of reference data having different incubation time attribute representing different incubation time.

2. The system of claim 1, wherein the computer system is configured for receiving data representing a determined parameter for a sample and receiving data representing an associated incubation time for said sample and wherein the computer system is programmed for
correlating the incubation time for said sample to said at least two sets of reference data and determining data representing a best fit standard curve correlated to said incubation time for said sample,
correlating the data representing the determined parameter to the data representing the best fit standard curve and determine data representing the target enzyme activity, and
transmitting said data representing the target enzyme activity to a display.

3. The system of claim 2, wherein the best fit standard curve is a calculated best fit standard curve obtained by performing a regression between two or more of said sets of reference data stored on said memory.

4. The system of any one of claims 1 to claim 3, wherein the target enzyme is a Hydrolase, a Lyase, a Ligase, a Lipase, a protease, a cellulose and/or an amylase.

5. The system of any one of claims 1 to claim 4, wherein the target enzyme is a degradative enzyme capable of degrading and/or digestion the substrate, such as glycoside hydrolase (e.g. amylase), protease, lipases and/or cellulase, amylase.

6. The system of any one of claims 1 to claim 5, wherein the substrate comprises a biopolymer such as a polymer comprising biomolecules selected from polynucleotides, polypeptides, polysaccharides or any combinations thereof, wherein the biopolymer optionally is crosslinked.

7. The system of any one of claims 1 to claim 6, wherein the substrate is a dyed substrate, a chromogenic substrate and/or a chromophore labelled substrate.

8. The system of any one of claims 1 to claim 7, wherein the parameter is an optical parameter and the reader is an optical reader, such as a spectroscope.

9. The system of any one of claims 2 to claim 8, wherein the computer system is configured for receiving at least a portion of said data representing the associated incubating time for said sample from a user via a user interface.

10. The system of any one of claims 2 to claim 9, wherein the computer system is configured for receiving at least a portion of said data representing the associated incubation time for said sample from the reader.

11. The system of any one of claims 2 to claim 10, wherein the computer system is configured for receiving said data representing the determined parameter for the sample from a user via a user interface and/or from the reader.

12. The system of any one of claims 1 to claim 11, wherein the computer system is storing at least three sets of reference data, such as 5 sets of reference data, each set of reference data comprises data representing a standard curve for said parameter associated to said enzymatic actions of said target enzyme involving said substrate correlated to an incubation time and an incubation time attribute representing said incubation time, wherein said sets of reference data having different incubation time attribute representing different incubation time.

13. The system of any one of claims 1 to claim 12, wherein each set of reference data comprises at least two data pairs of enzyme activity value versus parameter value, preferably each set of reference data comprises at least three data pairs, such as at least 5 data pairs, such as at least 8 data pairs.

14. The system of any one of claims 1 to claim 13, wherein said sets of reference data comprises at least two sets of reference data having different incubation time attribute representing incubation time that differs with at least about 30 seconds, such as at least about 1 minute, such as at least about 5 minutes, such as at least about 10 minutes.

15. The system of any one of claims 1 to claim 14, wherein the system is configured for obtaining the sample by extraction of a biological at least partly solid material, such as grain material and wherein the system further comprises
an extraction container,
an extraction buffer for extracting the preselected target enzyme,
container, such as a filter vial with a corresponding independent filter piston or a microcentrifuge tube.

16. The system of any one of claims 1-15, wherein said at least two sets of reference data represents a standard curve associated to a selected substrate.

17. The system of any one of claims 1-16, wherein said substrate is a dyed and/or chromogenic substrate.

18. The system of any one of claims 1-17, wherein said optical reader is configured for reading at least one wavelength absorbable by a dye of said substrate.

19. A method of determining activity of a target enzyme in a sample, the method comprises
bringing the sample in physical contact with a substrate for the target enzyme,
incubating the sample with the substrate for an actual incubating time,
determining a parameter value associated to enzymatic actions of said target enzyme involving said substrate,
correlating the actual incubation time for said sample to at least two sets of reference data,
determining a best fit standard curve correlated to said actual incubation time for said sample, and
correlating the determined parameter value to the best fit standard curve and determine the target enzyme activity,
wherein each of said at least two sets of reference data comprises data representing a standard curve for said parameter associated to said enzymatic actions of said target enzyme involving said substrate as a function of enzyme activity and correlated to an incubation time.

20. The method of claim 19, wherein the dyed and/or chromogenic substrate is specific for the target enzyme.

21. The method of claim 19 or claim 20, wherein the substrate is a gelled biopolymer substrate comprising cross-linked polymeric biomolecules selected from polynucleotides, polypeptides, polysaccharides or any combinations thereof, preferably the dyed and/or chromogenic substrate is an aerogel or a xerogel.

22. The method of any one of claims 19-21, wherein the method further comprises generating at least two sets of reference data, the method comprises generating each set of reference data by
providing a plurality of reference samples with different and known enzyme activity of the target enzyme,
providing a selected reference substrate for each reference sample,
subjecting each of the respective reference samples to the respective selected reference substrates for a preselected reference incubation time,
determining for each sample an a parameter value associated to enzymatic actions of said target enzyme involving said reference substrate,
performing a regression including points of respective pairs of determined absorbance parameter and corresponding, known enzyme activity to provide a linear standard curve.
wherein the preselected reference curve for generation of said respective sets of reference curves, differs from each other.

23. The method of claim 22, wherein the selected reference substrate is identical for the sample and for all reference samples.

24. The method of claim 22, wherein the plurality of material reference samples with different and known enzyme activity of the target enzyme comprises at least 3 reference samples, such as at least 5 reference samples, such as at least 8 reference samples.

25. The method of claim 22 or claim 23, wherein the selected reference substrate for the sample and for each reference sample is a dyed and/or chromogenic reference substrate.

26. The method of any one of claims 19-25, wherein the parameter value associated to enzymatic actions of said target enzyme involving said substrate is an absorbance parameter of dyed fragments released and/or generated from the substrate.

27. The method of any one of claimsclaim 22-26, wherein the actual incubation time is at least one minute, such as at least 2 minutes, such as 5 to 15 minutes.

28. The method of claim 27, wherein the preselected reference incubation time, for the generation of at least one of the sets of reference curves is from 5 minutes shorter to 5 minutes longer than the actual incubation time.
